(19) United States
(12) Patent Application Publication
(10) Pub. No.: US 2014/0004307 A1
(43) Pub. Date: Jan. 2, 2014

(54) Textured Fibrous Webs, Apparatus And Methods For Forming Textured Fibrous Webs

(71) Applicant: The Procter & Gamble Company, Cincinnati, OH (US)

(72) Inventor: Astrid Annette Sheehan, Cincinnati, OH (US)

(21) Appl. No.: 13/930,419

(22) Filed: Jun. 28, 2013

Related U.S. Application Data
(60) Provisional application No. 61/666,070, filed on Jun. 29, 2012.

(51) Int. Cl. A61K 8/02

(52) U.S. Cl. CPC A61K 8/0208
USPC 428/156; 264/257

(57) ABSTRACT

A fibrous web structure includes a first broad outer macroscopic surface and a second broad outer macroscopic surface opposite the first broad outer macroscopic surface thereby defining an absorbent fibrous region extending lengthwise in a longitudinal direction between the first and second broad outer macroscopic surfaces. The absorbent fibrous region has a thickness extending in a transverse direction that is perpendicular to the longitudinal direction. A formed fibrous feature defines a cavity extending at the first broad outer macroscopic surface. The formed fibrous feature has a wave formed of the fibrous region extending into a mouth of the cavity and a pocket defined by the cavity extending beyond the wave such that the wave overhangs the pocket.

### FIELD

The present invention is directed to textured fibrous webs and apparatus and processes for forming textured fibrous webs.

### BACKGROUND

Historically, various types of webs, such as nonwoven fibrous structures have been utilized as disposable substrates. The various types of webs used may differ in visual and tactile properties, usually due to the particular production processes used in their manufacture. In many cases, however, consumers of disposable webs suitable for use as wipes, such as baby wipes, demand strength, thickness, flexibility, texture and softness in addition to other functional attributes such as cleaning ability. Consumers often react to visual and tactile properties in their assessment of wipes.

Consumers often have a perception of the texture impression of a wipe based upon the appearance of the wipe itself, and, therefore, the perception is often subjective in nature. The texture of the wipe may provide visual signals to a consumer of product differentiation, strength, softness and cleaning efficacy. Additionally, wipes may have fluid uptake and retention properties such that they quickly acquire fluid during processing and remain wet during storage, and sufficient thickness, porosity, and texture to be effective in cleaning the soiled skin of a user.

The embossing of webs, such as paper webs, is known. Embossing of webs can provide improvements to the web such as increased bulk, improved water holding capacity, improved aesthetics and other benefits. Both single ply and multiple ply (or multi-ply) webs are known in the art and can be embossed. Multi-ply paper webs are webs that include at least two plies superimposed in face-to-face relationship to form a laminate.

During a typical embossing process, a web is fed through a nip formed between juxtaposed generally axially parallel rolls. Embossing elements on the rolls compress and/or deform the web. If a multi-ply product is being formed, two or more plies are fed through the nip and regions of each ply are brought into a contacting relationship with the opposing ply. The embossed regions of the plies may produce an aesthetic pattern and provide a means for joining and maintaining the plies in face-to-face contacting relationship.

Embossing is typically performed by one of two processes; knob-to-knob embossing or nested embossing. Knob-to-knob embossing typically consists of generally axially parallel rolls juxtaposed to form a nip between the embossing elements on opposing rolls. Nested embossing typically consists of embossing elements of one roll meshed between the embossing elements of the other roll. Examples of knob-to-knob embossing and nested embossing are illustrated by U.S. Pat. Nos. 3,414,459 issued Dec. 3, 1968 to Wells; 3,547,723 issued Dec. 15, 1970 to Gresham; 3,556,907 issued Jan. 19, 1971 to Nystrand; 3,708,366 issued Jan. 2, 1973 to Donnelly; 3,738,905 issued Jun. 12, 1973 to Thomas; 3,867,225 issued Feb. 18, 1975 to Nystrand; 4,483,728 issued Nov. 20, 1984 to Bauernfeind; 5,468,323 issued Nov. 21, 1995 to McNeil; 6,086,715 issued Jun. 11, 2000 to McNeil; 6,277,466 Aug. 21, 2001; 6,395,133 issued May 28, 2002 and 6,846,172 B2 issued to Vaughn et al. on Jan. 25, 2005.

Another type of embossing, deep-nested embossing, has been developed and used to provide unique characteristics to the embossed web. Deep-nested embossing refers to embossing that utilizes paired emboss elements, wherein the protrusions from the different embossing elements are coordinated such that the protrusions of one embossing element fit into the space between the protrusions of the other embossing element. Exemplary deep-nested embossing techniques are described in U.S. Pat. No. 5,686,168 issued to Laurent et al. on Nov. 11, 1997; U.S. Pat. No. 5,294,475 issued to McNeil on Mar. 15, 1994; U.S. patent application Ser. No. 11/059,986; U.S. patent application Ser. No. 10/700,131 and U.S. Patent Provisional Application Ser. No. 60/573,727.

### SUMMARY

In one embodiment, a fibrous web structure includes a first broad outer macroscopic surface and a second broad outer macroscopic surface opposite the first broad outer macroscopic surface thereby defining an absorbent fibrous region extending lengthwise in a longitudinal direction between the first and second broad outer macroscopic surfaces. The absorbent fibrous region has a thickness extending in a transverse direction that is perpendicular to the longitudinal direction. A formed fibrous feature defines a cavity at the first broad outer macroscopic surface. The formed fibrous feature has a wave formed of the fibrous region extending into a mouth of the cavity and a pocket defined by the cavity extending beyond the wave such that the wave overhangs the pocket.

In another embodiment, a fibrous web structure includes a first broad outer macroscopic surface and a second broad outer macroscopic surface opposite the first broad outer macroscopic surface thereby defining an absorbent fibrous region extending in a longitudinal direction between the first and second broad outer macroscopic surfaces. The absorbent fibrous region has a thickness extending in a transverse direction that is perpendicular to the longitudinal direction. A formed fibrous feature preform defines a cavity preform at the first broad outer macroscopic surface. The formed fibrous feature preform is configured to buckle under application of a compressive force to form a cavity including a wave formed of the fibrous region extending longitudinally into a mouth of the cavity and a pocket extending longitudinally beyond the wave of the cavity such that the wave overhangs the pocket.

In another embodiment, a method of forming a fibrous web structure including a first broad outer macroscopic surface and a second broad outer macroscopic surface opposite the first broad outer macroscopic surface thereby defining an absorbent fibrous region extending in a longitudinal direction between the first and second broad outer macroscopic surfaces is provided. The method includes forming a formed fibrous feature preform defining a cavity preform at the first broad outer macroscopic. A final formed fibrous feature defining a final cavity extending from the first broad outer macroscopic surface at a mouth of the final cavity is formed. The final formed fibrous feature has a wave formed of the fibrous region extending in the longitudinal direction into the mouth of the final cavity and a pocket defined by the final cavity extending in the longitudinal direction beyond the wave such that the wave overhangs the pocket.

In another embodiment, an apparatus for embossing a fibrous web structure including a first broad outer macroscopic surface and a second broad outer macroscopic surface opposite the first broad outer macroscopic surface thereby defining an absorbent fibrous region extending in a longitudinal direction between the first and second broad outer macroscopic surfaces is provided. The apparatus includes a cylindrical member having an outer periphery and a plurality of embossing projections extending outwardly from the outer periphery. Each embossing projection is shaped to form a formed fibrous feature preform in the fibrous structure defining a cavity preform at the first broad outer macroscopic surface at a mouth of the cavity. The formed fibrous feature preform, as formed by the embossing projection, is configured to buckle under application of a compressive force to form a final cavity including a wave formed of the fibrous region extending in the longitudinal direction into a mouth of the final cavity and a pocket extending in the longitudinal direction beyond the wave such that the wave overhangs the pocket.

### BRIEF DESCRIPTION OF THE DRAWINGS

The following detailed description will be more fully understood in view of the drawings in which:

FIG. 1 is a perspective view of an embodiment of a fibrous web structure including formed fibrous features;

FIG. 2 is a detailed view of the formed fibrous features of FIG. 1;

FIG. 3 is a detailed view of an embodiment of a formed fibrous feature preform for forming the formed fibrous features of FIG. 2;

FIG. 4 is a schematic view of an embodiment of an apparatus for embossing the fibrous web structure of FIG. 1;

FIG. 5 is a detail view of an embodiment of an embossing projection for use in the apparatus of FIG. 4;

FIGS. 6A and 6B are detail views of another embodiment of an embossing projection for use in the apparatus of FIG. 4;

FIG. 7 illustrates an embodiment of a process of deforming the preform formed fibrous features of FIG. 3 for forming the formed fibrous features of FIG. 2; and

FIG. 8 illustrates an embodiment of a winding apparatus for winding a continuous fibrous web structure.

The embodiment of the system shown in the drawings is illustrative in nature and is not intended to be limiting of the invention defined by the claims. Moreover, the features of the invention will be more fully apparent and understood in view of the detailed description.

### DETAILED DESCRIPTION

Embodiments described herein generally relate to fibrous web structures (e.g., wipes) that include formed fibrous features that define cavities for use in acquiring and retaining a substance from a surface or object which is animate or inanimate, and/or, application of a material to a surface or object which is animate or inanimate. For instance, wipes may be used for cleaning hard surfaces, such as floors. Wipes may also be used for human or animal cleansing or wiping such as anal cleansing, perineal cleansing, genital cleansing, and face and hand cleansing. Wipes may also be used for application of substances to the body, including but not limited to application of make-up, skin conditioners, ointments, and medications. They may also be used for cleaning or grooming of pets. Additionally, they may be used for general cleansing of surfaces and objects, such as household kitchen and bathroom surfaces, eyeglasses, exercise and athletic equipment, automotive surfaces, and the like.

“Fibrous web” or “fibrous web structure” as used herein means a structure that comprises one or more fibers. In one example, a fibrous web means an arrangement of interconnected fibers forming a web structure in order to perform a function. The fibrous web may be dry or wet. Suitable fibrous materials include woven and nonwoven materials, comprising natural fibers or synthetic fibers or combinations thereof. Examples of natural fibers may include cellulosic natural fibers, such as fibers from hardwood sources, softwood sources, or other non-wood plants. The natural fibers may comprise cellulose, starch and combinations thereof. The synthetic fibers can be any material, such as, but not limited to, those selected from the group consisting of polyesters (e.g., polyethylene terephthalate), polyolefins, polypropylenes, polyethylenes, polyethers, polyamides, polyesteramides, polyvinylalcohols, polyhydroxyalkanoates, polysaccharides and combinations thereof. Further, the synthetic fibers can be a single component (i.e., single synthetic material or mixture makes up entire fiber), bicomponent (i.e., the fiber is divided into regions, the regions including two or more different synthetic materials or mixtures thereof and may include co-extruded fibers and core and sheath fibers) and combinations thereof. Bi-component fibers can be used as a component fiber of the fibrous material, and/or they may be present to act as a binder for the other fibers present in the material. Any or all of the synthetic fibers may be treated before, during, or after manufacture to change any desired properties of the fibers.

“Non-woven fibrous web” as used herein is a fibrous web structure wherein fibers forming the fibrous structure are not orderly arranged by weaving and/or knitting the fibers together. The non-woven fibrous web structures may be disposable (i.e., typically thrown away after one or two uses—unlike clothes, rags, cloths, etc.).

“Fiber” as used herein means an elongate physical structure having an apparent length greatly exceeding its apparent diameter, i.e. a length to diameter ratio of at least about 10. Fibers having a non-circular cross-section and/or tubular shape may be used and the “diameter” in these cases may be considered to be the diameter of a circle having cross-sectional area equal to the cross-sectional area of the fiber. More specifically, as used herein, “fiber” refers to fibrous structure-making fibers. A variety of fibrous structure-making fibers may be used, such as, for example, naturally-occurring fibers or synthetic (human-made) fibers, or any other suitable fibers, and any combination thereof.

“Naturally-occurring fibers” as used herein means animal fibers, mineral fibers, plant fibers (such as wood fibers, trichomes and/or seed hairs) and mixtures thereof. Animal fibers may, for example, be selected from the group consisting of: wool, silk and other naturally-occurring protein fibers and mixtures thereof. The plant fibers may, for example, be obtained directly from a plant. Nonlimiting examples of suitable plants include wood, cotton, cotton linters, flax, sisal, abaca, hemp, hesperaloe, jute, bamboo, bagasse, kudzu, corn, sorghum, gourd, agave, loofah and mixtures thereof.

Wood fibers; often referred to as wood pulps include chemical pulps, such as kraft (sulfate) and sulfite pulps, as well as mechanical and semi-chemical pulps including, for example, groundwood, thermomechanical pulp, chemi-mechanical pulp (CMP), chemi-thermomechanical pulp (CTMP), neutral semi-chemical sulfite pulp (NSCS). Chemical pulps, however, may impart a tactile sense of softness to tissue sheets made therefrom. Pulps derived from both deciduous trees (hereinafter, also referred to as “hardwood”) and coniferous trees (hereinafter, also referred to as “softwood”) may be utilized. The hardwood and softwood fibers can be blended, or alternatively, can be deposited in layers to provide a stratified and/or layered web. U.S. Pat. No. 4,300,981 and U.S. Pat. No. 3,994,771 are incorporated herein by reference disclosing layering of hardwood and softwood fibers. Also applicable may be fibers derived from recycled paper, which may contain any or all of the above categories as well as other non-fibrous materials such as fillers and adhesives used to facilitate the original papermaking.

The wood fibers may be short (typical of hardwood fibers) or long (typical of softwood fibers). Nonlimiting examples of short fibers include fibers derived from a fiber source selected from the group consisting of Acacia, Eucalyptus, Maple, Oak, Aspen, Birch, Cottonwood, Alder, Ash, Chemy, Elm, Hickory, Poplar, Gum, Walnut, Locust, Sycamore, Beech, Catalpa, Sassafras, Gmelina, Albizia, Anthocephalus, and Magnolia. Nonlimiting examples of long fibers include fibers derived from Pine, Spruce, Fir, Tamarack, Hemlock, Cypress, and Cedar. Softwood fibers derived from the kraft process and originating from northern climates may be used.

In addition to the various wood fibers, other cellulosic fibers such as cotton linters, cotton and bagasse can be used in the fibrous structures of the present invention.

Synthetic (human-made) fibers (“non-naturally occurring fibers”), such as polymeric fibers, can also be used in the fibrous webs. Elastomeric polymers, polypropylene, polyethylene, polyester, polyolefin, polyvinyl alcohol and nylon, which are obtained from petroleum sources, can be used. In addition, polymeric fibers comprising natural polymers, which are obtained from natural sources, such as starch sources, protein sources and/or cellulose sources may be used in the fibrous webs. The synthetic fibers may be produced by any suitable methods.

“Sanitary tissue product” as used herein means a soft, low density (i.e. about 0.15 g/cm³) web useful as a wiping implement for post-urinary and post-bowel movement cleaning (toilet tissue), for otorhinolaryngological discharges (facial tissue), and multi-functional absorbent and cleaning uses (absorbent towels). The sanitary tissue product may be convolutedly wound upon itself about a core or without a core to form a roll of sanitary tissue product.

“Caliper” or “Sheet Caliper” as used herein refers to the macroscopic thickness of a single-ply fibrous web. As an example, a non-woven fibrous web may exhibit a sheet caliper of at least about 0.508 mm (20 mils) and/or at least about 0.762 mm (30 mils) and/or at least about 1.524 mm (60 mils).

“Absorbent” and “absorbency” as used herein means the characteristic of the fibrous structure which allows it to take up and retain fluids, particularly water, aqueous solutions and suspensions and waste fluids. In evaluating the absorbency of a fibrous web, not only is the absolute quantity of fluid a given amount of fibrous web will hold significant, but the rate at which the fibrous web will absorb the fluid is also.

“Liquid composition” and “lotion” are used interchangeably and refer to any liquid, including, but not limited to a pure liquid such as water, an aqueous solution, a colloid, an emulsion, a suspension, a solution and mixtures thereof. The term “aqueous solution” refers to a solution that is at least about 20%, at least about 40%, or even at least about 50% water by weight, and is no more than about 95%, or no more than about 90% water by weight.

“Pre-moistened” and “wet” are used interchangeably and refer to wipes which are moistened with a liquid composition prior to packaging in a generally moisture impervious container or wrapper. Such pre-moistened wipes, which can also be referred to as “wet wipes” and “towelettes”, may be suitable for use in cleaning babies, as well as older children and adults.

“Saturation loading” and “lotion loading” are used interchangeably and refer to the amount of liquid composition applied to the wipe. In general, the amount of liquid composition applied may be chosen in order to provide maximum benefits to the end product comprised by the wipe.

“Surface tension” refers to the force at the interface between a liquid composition and air. Surface tension is typically expressed in dynes per centimeter (dynes/cm).

“Surfactant” refers to materials which preferably orient toward an interface. Surfactants include nonionic surfactants; anionic surfactants; cationic surfactants; amphoteric surfactants, zwitterionic surfactants; and mixtures thereof.

“Machine Direction” or “MD” as used herein means the direction parallel to the flow of the fibrous structure through the papermaking machine and/or any type of fabric-making machine and/or product manufacturing equipment.

“Cross Machine Direction” or “CD” as used herein means the direction perpendicular to the machine direction in the same plane of the fibrous structure and/or paper product comprising the fibrous structure.

“Ply” or “Plies” as used herein means an individual fibrous structure optionally to be disposed in a substantially contiguous, face-to-face relationship with other plies, forming a multiple ply fibrous structure. It is also contemplated that a single fibrous structure can effectively form two “plies” or multiple “plies”, for example, by being folded on itself.

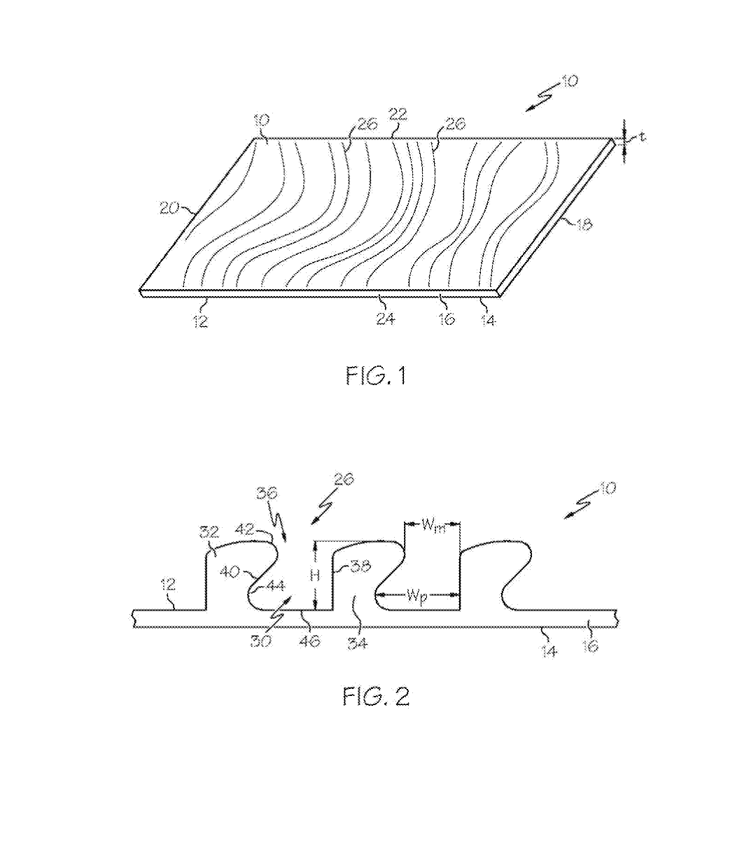

Referring to FIG. 1, a fibrous web structure 10 is illustrated in the form of a wipe, which may be cut or removed from a larger, continuous fibrous web structure. The fibrous web structure 10 includes a first broad outer macroscopic surface 12 and a second broad outer macroscopic surface 14. As used herein, the term “macroscopic” and its derivatives refer to structural features or elements that are readily visible and distinctly discernable to a human having a 20/20 vision when the perpendicular distance between the viewer's eye and the web is about 12 inches. An absorbent fibrous region 16 extends between the first and second macroscopic surfaces 12 and 14. Beyond wipes, it is contemplated that the fibrous web structure 10 may be used as a topsheet and/or backsheet of an adult incontinence pad (e.g., a feminine care pad) or for adult and/or baby diapers or pants. Additionally, the fibrous web structure may be used for absorbent article belts, as well as for cleaning substrates (e.g., Swiffer mop or wand refills).

The fibrous web structure 10 includes a leading edge 18, a trailing edge 20 and side edges 22 and 24 that extend, for example, longitudinally in the machine direction of the fibrous web structure 10 between the leading and trailing edges 18 and 20. In some embodiments, the leading edge 18 and trailing edge 20 may extend in the cross machine direction. The absorbent fibrous region 16 may have a thickness t that extends in a direction that is transverse to the longitudinal direction.

The fibrous web structure 10 may include a plurality of formed fibrous features 26 that extend continuously between the side edges 22 and 24. A used herein, the term “continuous” refers to an embossing feature that extends continuously along at least one path without a break or interruption. In other embodiments, one or more of the formed fibrous features 26 may be discontinuous. That is, a formed fibrous feature 26 may include multiple sections extending along a path with a break or interruption between the multiple sections. In the illustrated embodiment, the plurality of formed fibrous features 26 extend between the side edges 22 and 24 in a somewhat non-linear, wave-like path with adjacent formed fibrous features 26 being substantially parallel to one another. Other configurations are possible, for example, where adjacent formed fibrous features 26 are somewhat similarly oriented but not substantially parallel, cross paths and/or extend in the machine direction of the fibrous web structure 10.

Referring to FIG. 2, the formed fibrous features 26 each define a cavity 30 at the first broad outer macroscopic surface 12. Fibrous projections 32 and 34 formed of the fibrous region 16 are located at opposite leading and trailing sides of the cavity 30. The formed fibrous features 26 include a mouth 36 at the entrance of the cavity 30 with the leading fibrous projection 34 providing a leading wall 38 of the cavity 30 and the trailing fibrous projection 32 providing a trailing wall 40 of the cavity 30 that faces the leading wall 38. As can be seen by FIG. 2, the cavity 30 is non-square in shape and is defined by a wave portion 42 formed by the trailing wall 40 that extends into the mouth 36 of the cavity 36 and a pocket 44 that extends beyond the wave portion 42 in the machine direction such that the wave portion 42 of the trailing fibrous projection 32 overhangs the pocket 44. A longitudinally extending wall 46 extends from the leading wall 38 of the cavity 30 to the trailing wall 40 of the cavity 30. The longitudinal wall 46 provides a floor of the cavity 30 that closes or terminates the cavity 30 short of the second broad outer macroscopic surface 14 such that the cavity 30 is only partially enclosed, opening through the mouth 36.

In the exemplary embodiment of FIG. 2, the cavity 30 has a first width Wₘ measured in the longitudinal direction from the leading wall 38 to the wave portion 42 of the trailing wall 40.

The cavity 30 further has a second width Wₚ measured in the longitudinal direction from the leading wall 38 to an end of the pocket 44 at the trailing wall 40. In some embodiments, the first width Wₘ is different than the second width Wₚ. For example, the second width Wₚ may be greater than the first width Wₘ. Such differences in widths can provide the somewhat J-shaped cavity 30 illustrated by FIG. 2. While a J-shaped cavity 30 is illustrated, other shapes are possible. In some embodiments, a ratio of maximum height of the cavity 30 to the width Wₘ is at least about 1, such as at least about 1:0.5.

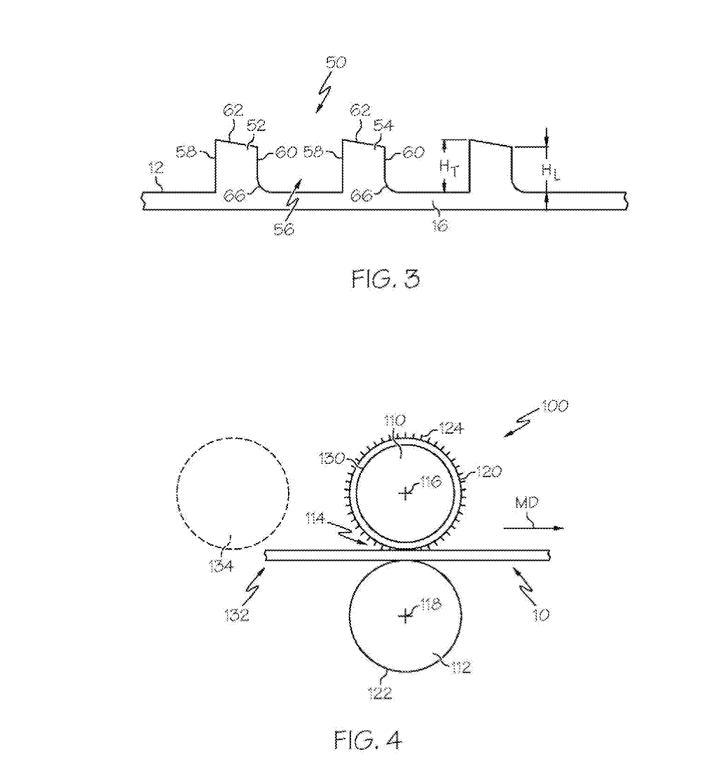

Referring to FIG. 3, the cavity 30 of FIG. 2 may be formed using a formed fibrous feature preform 50. As will be described below, the formed fibrous feature preform 50 may be formed using an embossing process using embossing projections shaped to form the formed fibrous feature preform 50. The formed fibrous feature preform 50 may include one or more fibrous projection preforms 52 and 54 formed of the fibrous region 16 and located at opposite leading and trailing sides of a cavity preform 56. In the illustrated example, each fibrous projection preform 52 and 54 includes a trailing wall 58, a leading wall 60 and a longitudinal wall 62 extending between the trailing wall 58 and the leading wall 60. Note that the trailing wall 58 of the fibrous projection preform 54 is the leading wall of the cavity preform 56 and the leading wall 60 of the fibrous projection preform 52 is the trailing wall of the cavity preform 56. The trailing walls 58 of the fibrous projection preforms 52 and 54 have a height H_{T} measured perpendicular to the first broad outer macroscopic surface 12 and the leading walls 60 of the fibrous projection preform 52 and 54 have a height H_{L} measured perpendicular to the first broad outer macroscopic surface 12. The heights H_{T} and H_{L} may be different. In some embodiments, such as the one shown, height H_{T} may be greater than height H_{L} or vice versa. In some embodiments, height H_{T} may be at least about 10 percent taller than H_{L}, such as at least about 25 percent taller than H_{L}, such as at least about 50 percent taller than H_{L}. In embodiments where the heights H_{T} and H_{L} are different, the longitudinal wall 62 may be at an angle to the horizontal. As one example, the longitudinal wall 62 may be at least about 15 degrees from the horizontal, such as about 45 degrees from the horizontal.

The leading walls 60 and/or trailing walls 58 of the fibrous projection preforms 52 and 54 may intersect the first broad outer macroscopic surface 12 at an angle or curve. In this embodiment, the leading walls 60 intersect the first broad outer macroscopic surface 12 at a curve 66. The radius of curvature of the curve 66 may be any suitable value, such as about 0.3 mm or more, such as about 0.5 mm or more, such as about 0.7 mm or more. In other embodiments, the leading and/or trailing walls 60 and 58 may be substantially perpendicular to the first broad outer macroscopic surface 12.

Referring now to FIG. 4, an apparatus 100 for embossing the fibrous web structure 10 is illustrated. The apparatus 100 includes a pair of rolls, first embossing roll 110 and second pressure roll 112. It should be noted that the embodiment shown in the figure is exemplary and other embodiments are certainly contemplated. For example, the embossing roll 110 and pressure roll 112 of the embodiment shown in FIG. 1 could be replaced with any other embossing members such as, for example, plates, cylinders or other equipment suitable for embossing webs. Further, additional equipment and steps that are not specifically described herein may be added to the apparatus and/or process. The embossing roll 110 and pressure roll 112 are disposed adjacent each other to provide a nip 114 that receives the fibrous web structure 10 (single or multiple plies may be delivered to the nip 114). The rolls 110 and 112 are generally configured so as to be rotatable on an axis, the axes 116 and 118, respectively, of the rolls 110 and 112 are typically generally parallel to one another. The apparatus 100 may be contained within an embossing device housing. Each roll 110 and 112 has an outer surface 120 and 122. The outer surface 120 of the embossing roll 110 may include a plurality of embossing projections 124. In some embodiments, the outer surface 122 of the pressure roll 112 may or may not include embossing projections. In the illustrated embodiment, the pressure roll 112 has a flat outer surface 122. The embossing roll 110 and pressure roll 112, including the surfaces 120 and 122 as well as the embossing projections 124, may be made out of any material suitable for the desired embossing process. Such materials include, without limitation, steel and other metals, ebonite, and hard rubber or a combination thereof. In some embodiments, a sleeve 130 including the embossing projections 124 may be applied to the embossing roll 110 and the sleeve may or may not be formed of a material (e.g., plastic or rubber) that is different than material (e.g., metal) forming the embossing roll 110. As shown in FIG. 4, the embossing roll 110 and the pressure roll 112 together provide the nip 114 through which a continuous fibrous web structure 132 (e.g., from a roll 134) can pass through the nip 114 in the machine direction MD.

As an alternative to embossing, FIG. 4 may represent a hydromolding process where a water jet is placed outside roll 110 and a vacuum is connected to roll 122 for drainage. Water or some other liquid may supply the pressure against the continuous fibrous web structure 132 for forming the formed fibrous features 26.

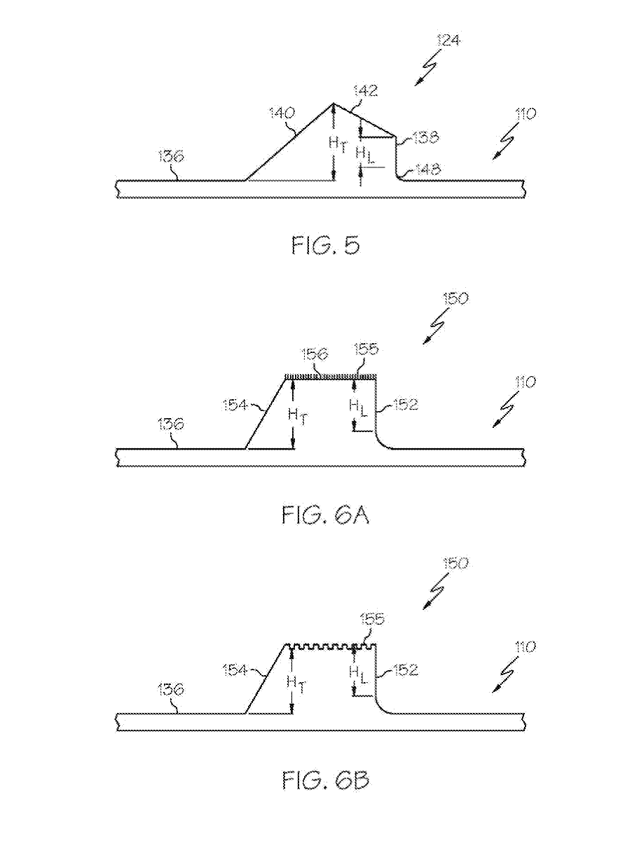

Referring to FIG. 5, an enlarged view of the embossing projections 124 of the embossing roll 110 is illustrated. Each embossing projection 124 extends outwardly from a periphery 136 of the embossing roll 110 and includes a leading wall 138, a trailing wall 140 and a longitudinal wall 142 extending between the leading and trailing walls 138 and 140. Of course, the terms “leading” and “trailing” depend on the direction of rotation of the embossing roll 110 and it should be noted that the leading wall 138 may become the trailing wall and the trailing wall 140 may become the leading wall 138. The trailing walls 140 of the embossing projections 124 have a height H_{T} measured perpendicular to the periphery 136 and the leading walls 138 of the embossing projections 124 have a height H_{L} measured perpendicular to the periphery 136. The heights H_{T} and H_{L} may be different. In some embodiments, such as the one shown, height H_{T} may be greater than height H_{L} or vice versa. In some embodiments, height H_{T} may be at least about 10 percent taller than H_{L}, such as at least about 25 percent taller than H_{L}, such as at least about 50 percent taller than H_{L}. In embodiments where the heights H_{T} and H_{L} are different, the longitudinal wall 142 may be at an angle to the horizontal. As one example, the longitudinal wall 142 may be at least about 15 degrees from the horizontal, such as about 45 degrees from the horizontal.

The leading walls 138 and/or trailing walls 140 of the embossing projections 124 may intersect the periphery 136 at an angle or curve. In this embodiment, the leading walls 138 intersect the periphery 136 at a curve 148. The radius of curvature of the curve 148 may be any suitable value, such as about 0.3 mm or more, such as about 0.5 mm or more, such as about 0.7 mm or more. In other embodiments the leading and/or trailing walls 138 and 140 of the embossing projections 124 may be substantially perpendicular with the periphery 136.

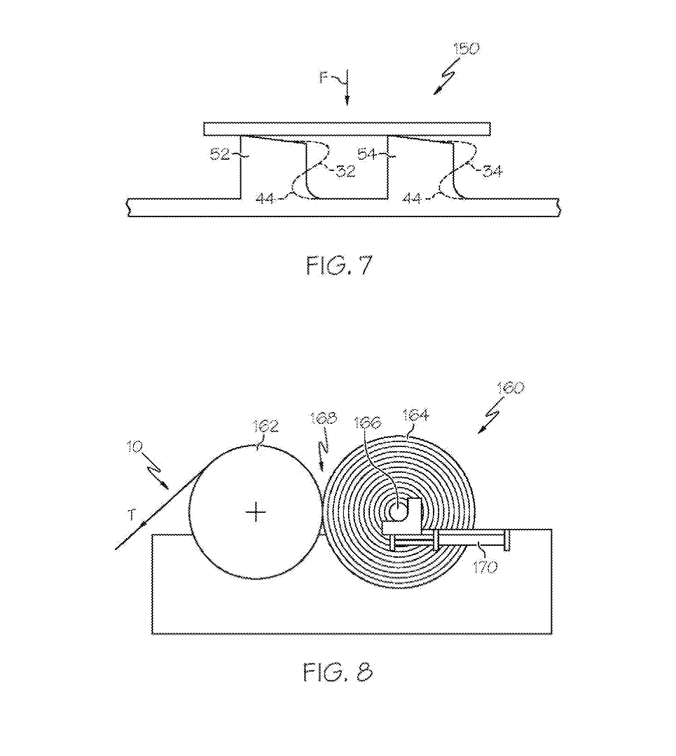

The embossing projections 124 are shaped to form the embossing feature preforms including the fibrous projection preforms formed of the fibrous region and the cavity preforms (see e.g., FIG. 3). While embossing projections 124 are illustrated by FIG. 5, other embossing projection shapes are contemplated. For example, referring to FIGS. 6A and 6B, another embossing projection 150 includes a leading wall 152, a trailing wall 154 and a longitudinal wall 156 extending between the leading and trailing walls 152 and 154. The trailing walls 154 of the embossing projections 150 have a height H_{T} measured perpendicular to the periphery 136 and the leading walls 152 of the embossing projections 150 have a height H_{L} measured perpendicular to the periphery 136. The heights H_{T} and H_{L} may be the same. In embodiments where the heights H_{T} and H_{L} are the same, the longitudinal wall 156 may be substantially horizontal. In some embodiments, the longitudinal wall 156 may include an array of microfeatures 155 (e.g., in the form of projections or pattern of projections) extending outwardly from the longitudinal wall 156. The microfeatures 155 may be used to impart a microtexture within the cavity 30 of the fibrous web structure 10.

Referring to FIG. 7, once the formed fibrous feature preform 50 is formed using the apparatus 100 and the embossing projections 124, the formed fibrous features 26 are formed by deforming the fibrous projection preforms 52 and 54. In some embodiments, a compressive force F may be applied against the fibrous projection preforms 52 and 54. The shapes of the fibrous projection preforms 52 and 54 and the cavity preform 56 including the radius 66 cause the fibrous projection preforms 52 and 54 to buckle and form the formed fibrous features 26 of FIG. 2 including the fibrous projections 32 and 34 and the cavity 30 including the pocket 44.

The compressive force F may be applied by any suitable method. As one example, the fibrous web structure 10 including the formed fibrous feature preform 50 may be delivered between two pressure rolls that apply the compressive force F. As another example, the fibrous web structure 10 may be placed on a table or other support structure and a press may apply the compressive force. In some embodiments, the compressive force F may be applied while the fibrous web structure 10 is in a roll form.

Referring to FIG. 8, one exemplary winding apparatus 160 includes a winding drum 162 and a winding roll 164 including a core 166 about which the continuous fibrous web structure 10 is wound. A nip 168 is formed between the winding drum 162 and the winding roll 164. Pressure in the nip 168 can be controlled or adjusted using an actuator 170, such as a hydraulic or pneumatic cylinder. Tension T is maintained in the fibrous web structure 10 as the fibrous web structure 10 enters the nip 168 and is wound about the core 166. Without wishing to be bound by theory, as the continuous web structure 10 is wound about the core 166, tension builds within the winding roll 164, which may be referred to as in-wound tension. A number of factors may affect the in-wound tension and compressive forces within the winding roll. These factors may include the web tension, the nip pressure and the winding torque. The continuous fibrous web structure 10 may be rolled to provide compressive forces of at least about 0.1 psi, such as between about 0.4 psi and about 0.8 psi to cause the fibrous projection preforms 52 and 54 to buckle and form the formed fibrous features 26 of FIG. 2 including the fibrous projections 32 and 34 and the cavity 30 including the pocket 44. In some embodiments, the compressive forces may be less than 0.1 psi. Depending on location within the winding roll 164, the compressive forces may be between about 0 psi and between about 0.4 psi and about 0.8 psi.

Fibrous Web Structure

The fibrous web structure 10 may consist of any web, mat, or batt of loose fibers, disposed in relationship with one another in some degree of alignment, such as might be produced by carding, air-laying, spunbonding, and the like. The fibrous web may be a precursor to a nonwoven molded fibrous structure. The fibers of the fibrous web, and subsequently the nonwoven molded fibrous structure, may be any natural, cellulosic, and/or wholly synthetic material. Examples of natural fibers may include cellulosic natural fibers, such as fibers from hardwood sources, softwood sources, or other non-wood plants. The natural fibers may comprise cellulose, starch and combinations thereof. Non-limiting examples of suitable cellulosic natural fibers include, but are not limited to, wood pulp, typical northern softwood Kraft, typical southern softwood Kraft, typical CTMP, typical deinked, corn pulp, acacia, eucalyptus, aspen, reed pulp, birch, maple, radiata pine and combinations thereof. Other sources of natural fibers from plants include, but are not limited to, albardine, esparto, wheat, rice, corn, sugar cane, papyrus, jute, reed, sabia, raphia, bamboo, sidal, kenaf, abaca, sunn, rayon (also known as viscose), lyocell, cotton, hemp, flax, ramie and combinations thereof. Yet other natural fibers may include fibers from other natural non-plant sources, such as, down, feathers, silk, cotton and combinations thereof. The natural fibers may be treated or otherwise modified mechanically or chemically to provide desired characteristics or may be in a form that is generally similar to the form in which they can be found in nature. Mechanical and/or chemical manipulation of natural fibers does not exclude them from what are considered natural fibers with respect to the development described herein.

The synthetic fibers can be any material, such as, but not limited to, those selected from the group consisting of polyesters (e.g., polyethylene terephthalate), polyolefins, polypropylenes, polyethylenes, polyethers, polyamides, polyesteramides, polyvinylalcohols, polyhydroxyalkanoates, polysaccharides, and combinations thereof. Further, the synthetic fibers can be a single component (i.e., single synthetic material or mixture makes up entire fiber), bicomponent (i.e., the fiber is divided into regions, the regions including two or more different synthetic materials or mixtures thereof and may include co-extruded fibers and core and sheath fibers) and combinations thereof. It is also possible to use bicomponent fibers. These bicomponent fibers can be used as a component fiber of the structure, and/or they may be present to act as a binder for the other fibers present in the fibrous structure. Any or all of the synthetic fibers may be treated before, during, or after the process to change any desired properties of the fibers. For example, in certain embodiments, it may be desirable to treat the synthetic fibers before or during processing to make them more hydrophilic, more wettable, etc.

In certain embodiments, it may be desirable to have particular combinations of fibers to provide desired characteristics. For example, it may be desirable to have fibers of certain lengths, widths, coarseness or other characteristics combined in certain layers or separate from each other. The fibers may be of virtually any size and may have an average length from about 1 mm to about 60 mm. Average fiber length refers to the length of the individual fibers if straightened out. The fibers may have an average fiber width of greater than about 5 micrometers. The fibers may have an average fiber width of from about 5 micrometers to about 50 micrometers. The fibers may have a coarseness of greater than about 5 mg/100 m. The fibers may have a coarseness of from about 5 mg/100 m to about 75 mg/100 m.

The fibers may be circular in cross-section, dog bone shaped, delta (i.e., triangular cross-section), tri-lobal, ribbon, or other shapes typically produced as staple fibers. Likewise, the fibers can be conjugate fibers, such as bicomponent fibers. The fibers may be crimped, and may have a finish, such as a lubricant, applied.

The fibrous web of an embodiment may have a basis weight of between about 30, 40 or 45 gsm and about 50, 55, 60, 65, 70, or 75 gsm. Fibrous webs may be available from the J.W. Suominen Company of Finland, and sold under the FIBRELLA trade name. For example, FIBRELLA 3100 and FIBRELLA 3160 have been found to be useful as fibrous webs. FIBRELLA 3100 is a 62 gsm nonwoven web comprising 50% 1.5 denier polypropylene fibers and 50% 1.5 denier viscose fibers. FIBRELLA 3160 is a 58 gsm nonwoven web comprising 60% 1.5 denier polypropylene fibers and 40% 1.5 denier viscose fibers. In both of these commercially available fibrous webs, the average fiber length is about 38 mm. Additional fibrous webs available from Suominen may include a 62 gsm nonwoven web comprising 60% polypropylene fibers and 40% viscose fibers; a fibrous web comprising a basis weight from about 50 or 55 to about 58 or 62 and comprising 60% polypropylene fibers and 40% viscose fibers; and a fibrous web comprising a basis weight from about 62 to about 70 or 75 gsm. The latter fibrous web may comprise 60% polypropylene fibers and 40% viscose fibers.

The fibrous structure may take a number of different forms. The fibrous structure may comprise 100% synthetic fibers or may be a combination of synthetic fibers and natural fibers. In one embodiment, the fibrous structure may include one or more layers of a plurality of synthetic fibers mixed with a plurality of natural fibers. The synthetic fiber/natural fiber mix may be relatively homogeneous in that the different fibers may be dispersed generally randomly throughout the layer. The fiber mix may be structured such that the synthetic fibers and natural fibers may be disposed generally non-randomly. In one embodiment, the fibrous structure may include at least one layer comprising a plurality of natural fibers and at least one adjacent layer comprising a plurality of synthetic fibers. In another embodiment, the fibrous structure may include at least one layer that includes a plurality of synthetic fibers homogeneously mixed with a plurality of natural fibers and at least one adjacent layer that includes a plurality of natural fibers. In an alternate embodiment, the fibrous structure may include at least one layer that includes a plurality of natural fibers and at least one adjacent layer that may comprise a mixture of a plurality of synthetic fibers and a plurality of natural fibers in which the synthetic fibers and/or natural fibers may be disposed generally non-randomly. Further, one or more of the layers of mixed natural fibers and synthetic fibers may be subjected to manipulation during or after the formation of the fibrous structure to disperse the layer or layers of mixed synthetic and natural fibers in a predetermined pattern or other non-random pattern.

The fibrous structure may further include binder materials. The fibrous structure may include from about 0.01% to about 1%, 3%, or 5% by weight of a binder material selected from a group of permanent wet strength resins, temporary wet strength resins, dry strength resins, retention aid resins and combinations thereof.

If permanent wet strength is desired, the binder materials may be selected from the group of polyamide-epichlorohydrin, polyacrylamides, styrene-butadiene latexes, insolubilized polyvinyl alcohol, ureaformaldehyde, polyethyleneimine, chitosan polymers and combinations thereof.

If temporary wet strength is desired, the binder materials may be starch based. Starch based temporary wet strength resins may be selected from the group of cationic dialdehyde starch-based resin, dialdehyde starch and combinations thereof. The resin described in U.S. Pat. No. 4,981,557, issued Jan. 1, 1991 to Bjorkquist may also be used.

If dry strength is desired, the binder materials may be selected from the group of polyacrylamide, starch, polyvinyl alcohol, guar or locust bean gums, polyacrylate latexes, carboxymethyl cellulose and combinations thereof.

A latex binder may also be utilized. Such a latex binder may have a glass transition temperature from about 0° C., −10° C., or −20° C. to about −40° C., −60° C., or −80° C. Examples of latex binders that may be used include polymers and copolymers of acrylate esters, referred to generally as acrylic polymers, vinyl acetate-ethylene copolymers, styrene-butadiene copolymers, vinyl chloride polymers, vinylidene chloride polymers, vinyl chloride-vinylidene chloride copolymers, acrylo-nitrile copolymers, acrylic-ethylene copolymers and combinations thereof. The water emulsions of these latex binders usually contain surfactants. These surfactants may be modified during drying and curing so that they become incapable of rewetting.

Methods of application of the binder materials may include aqueous emulsion, wet end addition, spraying and printing. At least an effective amount of binder may be applied to the fibrous structure. Between about 0.01% and about 1.0%, 3.0% or 5.0% may be retained on the fibrous structure, calculated on a dry fiber weight basis. The binder may be applied to the fibrous structure in an intermittent pattern generally covering less than about 50% of the surface area of the structure. The binder may also be applied to the fibrous structure in a pattern to generally cover greater than about 50% of the fibrous structure. The binder material may be disposed on the fibrous structure in a random distribution. Alternatively, the binder material may be disposed on the fibrous structure in a non-random repeating pattern.

Additional information relating to the fibrous structure may be found in U.S. Patent Application No. 2004/0154768, filed by Trokhan et al. and published Aug. 12, 2004, US Patent Application No. 2004/0157524, filed by Polat et al. and published Aug. 12, 2004, U.S. Pat. No. 4,588,457, issued to Crenshaw et al., May 13, 1986, U.S. Pat. No. 5,397,435, issued to Ostendorf et al., Mar. 14, 1995 and U.S. Pat. No. 5,405,501, issued to Phan et al., Apr. 11, 1995.

The fibrous structure, as described herein, may be utilized to form a substrate. The fibrous structure may continue to be processed in any method to convert the fibrous structure to a substrate having at least one molded element. This may include, but is not limited to, slitting, cutting, perforating, folding, stacking, interleaving, lotioning and combinations thereof.

The material from which a substrate is made should be strong enough to resist tearing during manufacture and normal use, yet still provide softness to the user's skin, such as a child's tender skin. Additionally, the material should be at least capable of retaining its form for the duration of the user's cleansing experience.

Substrates may be generally of sufficient dimension to allow for convenient handling. Typically, the substrate may be cut and/or folded to such dimensions as part of the manufacturing process. The substrate may be cut into individual portions so as to provide separate wipes which are often stacked and interleaved in consumer packaging. Suitably, the separate wipes may have a length between about 100 mm and about 250 mm and a width between about 140 mm and about 250 mm. In one embodiment, the separate wipe may be about 200 mm long and about 180 mm wide.

The material of the substrate may generally be soft and flexible, potentially having a structured surface to enhance its performance. The substrate may include laminates of two or more materials. Commercially available laminates, or purposely built laminates are contemplated. The laminated materials may be joined or bonded together in any suitable fashion, such as, but not limited to, ultrasonic bonding, adhesive, glue, fusion bonding, heat bonding, thermal bonding, hydroentangling and combinations thereof. In another alternative embodiment the substrate may be a laminate comprising one or more layers of nonwoven materials and one or more layers of film. Examples of such optional films, include, but are not limited to, polyolefin films, such as, polyethylene film. An illustrative, but non-limiting example of a nonwoven sheet member which is a laminate of a 16 gsm nonwoven polypropylene and a 0.8 mm 20 gsm polyethylene film.

The substrate materials may also be treated to improve the softness and texture thereof. The substrate may be subjected to various treatments, such as, but not limited to, physical treatment, such as ring rolling, as described in U.S. Pat. No. 5,143,679; structural elongation, as described in U.S. Pat. No. 5,518,801; consolidation, as described in U.S. Pat. Nos. 5,914,084, 6,114,263, 6,129,801 and 6,383,431; stretch aperturing, as described in U.S. Pat. Nos. 5,628,097, 5,658,639 and 5,916,661; differential elongation, as described in WO Publication No. 2003/0028165A1; and other solid state formation technologies as described in U.S. Publication No. 2004/0131820A1 and U.S. Publication No. 2004/0265534A1, zone activation, and the like; chemical treatment, such as, but not limited to, rendering part or all of the substrate hydrophobic, and/or hydrophilic, and the like; thermal treatment, such as, but not limited to, softening of fibers by heating, thermal bonding and the like; and combinations thereof.

The substrate may have a basis weight of at least about 30 grams/m². The substrate may have a basis weight of at least about 40 grams/m². In one embodiment, the substrate may have a basis weight of at least about 45 grams/m². In another embodiment, the substrate basis weight may be less than about 75 grams/m². In another embodiment, substrates may have a basis weight between about 40 grams/m² and about 75 grams/m², and in yet another embodiment a basis weight between about 40 grams/m² and about 65 grams/m². The substrate may have a basis weight between about 30, 40, or 45 and about 50, 55, 60, 65, 70 or 75 grams/m².

A suitable substrate may be a carded nonwoven comprising a 40/60 blend of viscose fibers and polypropylene fibers having a basis weight of 58 grams/m² as available from Suominen of Tampere, Finland as FIBRELLA 3160. Another suitable material for use as a substrate may be SAWATEX 2642 as available from Sandler AG of Schwarzenbach/Salle, Germany. Yet another suitable material for use as a substrate may have a basis weight of from about 50 grams/m² to about 60 grams/m² and have a 20/80 blend of viscose fibers and polypropylene fibers. The substrate may also be a 60/40 blend of pulp and viscose fibers. The substrate may also be formed from any of the following fibrous webs such as those available from the J.W. Suominen Company of Finland, and sold under the FIBRELLA trade name. For example, FIBRELLA 3100 is a 62 gsm nonwoven web comprising 50% 1.5 denier polypropylene fibers and 50% 1.5 denier viscose fibers. In both of these commercially available fibrous webs, the average fiber length is about 38 mm. Additional fibrous webs available from Suominen may include a 62 gsm nonwoven web comprising 60% polypropylene fibers and 40% viscose fibers; a fibrous web comprising a basis weight from about 50 or 55 to about 58 or 62 and comprising 60% polypropylene fibers and 40% viscose fibers; and a fibrous web comprising a basis weight from about 62 to about 70 or 75 gsm. The latter fibrous web may comprise 60% polypropylene fibers and 40% viscose fibers.

Any other suitable materials may be used for forming the fibrous web structure 10. Papermaking fibers may be useful in forming the fibrous web structure and include cellulosic fibers commonly known as wood pulp fibers. Applicable wood pulps include chemical pulps, such as Kraft, sulfite, and sulfate pulps, as well as mechanical pulps including, for example, groundwood, thermomechanical pulp and chemically modified thermomechanical pulp. Chemical pulps, however, may be preferred in certain embodiments since they may impart a superior tactile sense of softness to tissue sheets made therefrom. Pulps derived from both deciduous trees (hereinafter, also referred to as “hardwood”) and coniferous trees (hereinafter, also referred to as “softwood”) may be utilized. The hardwood and softwood fibers can be blended, or alternatively, can be deposited in layers to provide a stratified web. U.S. Pat. Nos. 4,300,981 and 3,994,771 disclose layering of hardwood and softwood fibers. Also applicable are fibers derived from recycled paper, which may contain any or all of the above categories as well as other non-fibrous materials such as fillers and adhesives used to facilitate the original papermaking. In addition to the above, fibers and/or filaments made from polymers, specifically hydroxyl polymers may be used. Nonlimiting examples of suitable hydroxyl polymers include polyvinyl alcohol, starch, starch derivatives, chitosan, chitosan derivatives, cellulose derivatives, gums, arabinans, galactans and mixtures thereof.

The papermaking fibers may include fibers derived from wood pulp. Other natural fibrous pulp fibers, such as cotton linters, bagasse, wool fibers, silk fibers, etc., can be utilized. Synthetic fibers, such as rayon, polyethylene and polypropylene fibers, may also be utilized in combination with natural cellulosic fibers. One exemplary polyethylene fiber which may be utilized is Pulpex®, available from Hercules, Inc. (Wilmington, Del.).

Representative examples of paper substrates can be found in U.S. Pat. No. 4,629,643 issued to Curro et al. on Dec. 16, 1986; U.S. Pat. No. 4,609,518 issued to Curro et al. on Sep. 2, 1986; U.S. Pat. No. 4,603,069 issued to Haq et al. on Jul. 29, 1986; U.S. Patent Publications 2004/0154768 A1 published to Trokhan et al. on Aug. 12, 2004; 2004/0154767 A1 published to Trokhan et al. on Aug. 12, 2004; 2003/0021952 A1 published to Zink et al. on Jan. 30, 2003; and 2003/0028165 A1 published to Curro et al. on Feb. 6, 2003.

The paper product substrate may comprise any paper product known in the industry. Embodiments of these substrates may be made according U.S. Pat. Nos. 4,191,609 issued Mar. 4, 1980 to Trokhan; 4,300,981 issued to Carstens on Nov. 17, 1981; 4,514,345 issued to Johnson et al. on Apr. 30, 1985; 4,528,239 issued to Trokhan on Jul. 9, 1985; 4,529,480 issued to Trokhan on Jul. 16, 1985; 4,637,859 issued to Trokhan on Jan. 20, 1987; 5,245,025 issued to Trokhan et al. on Sep. 14, 1993; 5,275,700 issued to Trokhan on Jan. 4, 1994; 5,328,565 issued to Rasch et al. on Jul. 12, 1994; 5,334,289 issued to Trokhan et al. on Aug. 2, 1994; 5,364,504 issued to Smurkowski et al. on Nov. 15, 1995; 5,527,428 issued to Trokhan et al. on Jun. 18, 1996; 5,556,509 issued to Trokhan et al. on Sep. 17, 1996; 5,628,876 issued to Ayers et al. on May 13, 1997; 5,629,052 issued to Trokhan et al. on May 13, 1997; 5,637,194 issued to Ampulski et al. on Jun. 10, 1997; 5,411,636 issued to Hermans et al. on May 2, 1995; 6,017,417 issued to Wendt et al. on Jan. 25, 2000; 5,746,887 issued to Wendt et al. on May 5, 1998; 5,672,248 issued to Wendt et al. on Sep. 30, 1997; and U.S. Patent Application 2004/0192136A1 published in the name of Gusky et al. on Sep. 30, 2004.

The paper substrates may be manufactured via a wet-laid papermaking process where the resulting web is through-air-dried or conventionally dried. Optionally, the substrate may be foreshortened by creping, by wet microcontraction or by any other means. Creping and/or wet microcontraction are disclosed in U.S. Pat. No. 6,048,938 issued to Neal et al. on Apr. 11, 2000; U.S. Pat. No. 5,942,085 issued to Neal et al. on Aug. 24, 1999; U.S. Pat. No. 5,865,950 issued to Vinson et al. on Feb. 2, 1999; U.S. Pat. No. 4,440,597 issued to Wells et al. on Apr. 3, 1984; U.S. Pat. No. 4,191,756 issued to Sawdai on May 4, 1980; and U.S. Pat. No. 6,187,138 issued to Neal et al. on Feb. 13, 2001.

Conventionally pressed tissue paper and methods for making such paper are, for example, as described in U.S. Pat. No. 6,547,928 issued to Barnholtz et al. on Apr. 15, 2003. One suitable tissue paper is pattern densified tissue paper which is characterized by having a relatively high-bulk field of relatively low fiber density and an array of densified zones of relatively high fiber density. The high-bulk field is alternatively characterized as a field of pillow regions. The densified zones are alternatively referred to as knuckle regions. The densified zones may be discretely spaced within the high-bulk field or may be interconnected, either fully or partially, within the high-bulk field. Processes for making pattern densified tissue webs are disclosed in U.S. Pat. No. 3,301,746 issued to Sanford and Sisson on Jan. 31, 1967; U.S. Pat. No. 3,473,576, issued to Amneus on Oct. 21, 1969; U.S. Pat. No. 3,573,164 issued to Friedberg, et al. on Mar. 30, 1971; U.S. Pat. No. 3,821,068 issued to Salvucci, Jr. et al. on May 21, 1974; U.S. Pat. No. 3,974,025 issued to Ayers on Aug. 10, 1976; U.S. Pat. No. 4,191,609 issued to on Mar. 4, 1980; U.S. Pat. No. 4,239,065 issued to Trokhan on Dec. 16, 1980 and U.S. Pat. No. 4,528,239 issued to Trokhan on Jul. 9, 1985 and U.S. Pat. No. 4,637,859 issued to Trokhan on Jan. 20, 1987.

Uncompacted, non pattern-densified tissue paper structures are also contemplated and are described in U.S. Pat. No. 3,812,000 issued to Joseph L. Salvucci, Jr. and Peter N. Yiannos on May 21, 1974, and U.S. Pat. No. 4,208,459 issued to Henry E. Becker, Albert L. McConnell, and Richard Schutte on Jun. 17, 1980. Uncreped paper can also be subjected to the apparatus and method of the present invention. Suitable techniques for producing uncreped tissue are taught, for example, in U.S. Pat. No. 6,017,417 issued to Wendt et al. on Jan. 25, 2000; U.S. Pat. No. 5,746,887 issued to Wendt et al. on May 5, 1998; U.S. Pat. No. 5,672,248 issued to Wendt et al. on Sep. 30, 1997; U.S. Pat. No. 5,888,347 issued to Engel et al. on Mar. 30, 1999; U.S. Pat. No. 5,667,636 issued to Engel et al. on Sep. 16, 1997; U.S. Pat. No. 5,607,551 issued to Farrington et al. on Mar. 4, 1997 and U.S. Pat. No. 5,656,132 issued to Farrington et al. on Aug. 12, 1997.

Tissue-towel substrates may alternatively be manufactured via an air-laid making process. Typical airlaying processes include one or more forming chambers that are placed over a moving foraminous surface, such as a forming screen. For example, fibrous materials and particulate materials are introduced into the forming chamber and a vacuum source is employed to draw an airstream through the forming surface. The air stream deposits the fibers and particulate material onto the moving forming surface. Once the fibers are deposited onto the forming surface, an airlaid web substrate is formed. Once the web exits the forming chambers, the web is passed through one or more compaction devices which increases the density and strength of the web. The density of the web may be increased to between about 0.05 g/cc to about 0.5 g/cc. After compaction, the one or both sides of the web may optionally be sprayed with a bonding material, such as latex compositions or other known water-soluble bonding agents, to add wet and dry strength. If a bonding agent is applied, the web is typically passed through a drying apparatus. An example of one process for making such airlaid paper substrates is found in U.S. Patent Application 2004/0192136A1 filed in the name of Gusky et al. and published on Sep. 30, 2004.

The apparatus and method are not limited to any particular type of papermaking and/or converting equipment and can be operated at any suitable line speed. Certain exemplary papermaking and converting equipment are identified herein. Further, although not limited to any particular line speed, typical converting line speeds generally range between about 300 and about 700 meters per minute.

Other optional equipment may be used and/or processes may be performed on the web during its manufacture or after it is manufactured, as desired. These processes can be performed before or after the embossing method, as applicable. For example, in certain embodiments, it may be desirable to print on the web. It may also be desirable to register the printing to the emboss pattern. Exemplary methods for registering printing to the embossing pattern are described in more detail in U.S. Patent Application Publication No. 2004/0258887 A1 published Dec. 23, 2004 and 2004/0261639 A1 published Dec. 30, 2004. It may also be desirable to provide heat, moisture or steam to the web prior to the web being embossed. Exemplary suitable apparatuses and methods for providing steam to a web to be embossed are described in U.S. Pat. No. 4,207,143 issued to Glomb et al. on Jun. 10, 1980; U.S. Pat. No. 4,994,144 issued to Smith et al. on Feb. 19, 1991; U.S. Pat. No. 6,074,525 issued to Richards on Jun. 13, 2000 and U.S. Pat. No. 6,077,590 issued to Archer on Jun. 20, 2000. However any suitable apparatus and/or method for providing heat, moisture or steam to the web may be used, including the use of steam bars, airfoils, sprayers, steam chambers or any combination thereof.

Further, for paper webs, optional materials can be added to the aqueous papermaking furnish or the embryonic web to impart other desirable characteristics to the product or improve the papermaking process. Some examples of such materials may include softening agents, wet-strength agents, surfactants, fillers and other known additives or combinations thereof. Similarly, for non-paper webs, optional ingredients, coatings or processes can be used to provide the web with any particular desired characteristics and/or alter the base web's physical or chemical characteristics.

Soothing and/or Cleansing Composition

The substrate may further include a soothing and/or cleansing composition. The composition impregnating the substrate is commonly and interchangeably called lotion, soothing lotion, soothing composition, oil-in-water emulsion composition, emulsion composition, emulsion, cleaning or cleansing lotion or composition. All those terms are hereby used interchangeably. The composition may generally comprise the following optional ingredients: emollients, surfactants and/or an emulsifiers, soothing agents, rheology modifiers, preservatives, or more specifically a combination of preservative compounds acting together as a preservative system and water.

It is to be noted that some compounds can have a multiple function and that all compounds are not necessarily present in the composition of the invention. The composition may be a oil-in-water emulsion. The pH of the composition may be from about pH 3, 4 or 5 to about pH 7, 7.5, or 9.

Skin Agent

The substrate may further include a skin agent. The skin agent can be any suitable agent, including, for example, lotions, anhydrous coatings, surface treating compositions, nanotechnology agents, encapsulated time release agents, skin healants, anesthetics, analgesics, perfumes, such as long lasting or enduring perfumes, antibacterial agents, antiviral agents, botanical agents, disinfectants, pharmaceutical agents, film formers, dyes, inks, colorants, surfactants, absorbents, wet strength agents, deodorants, opacifiers, astringents, solvents, biological agents such as bacteria, viruses and their toxins, absorbent structure materials or mixtures thereof.

Surface Treating Composition

The substrate may comprise a surface treating composition. The surface treating composition may be a composition comprised of one or more surface treating agents that improves the tactile sensation of a surface of an absorbent structure as perceived by a user who holds the absorbent structure and rubs it across the area of skin. Such tactile perceivable softness can be characterized by, but is not limited to, friction, flexibility, and smoothness, as well as subjective descriptors, such as a feeling like lubricious, velvet, silk or flannel. The surface treating composition may or may not be transferable. In certain embodiments, the surface treating composition may be substantially non-transferable.

Examples of surface treating agents include but are not limited to at least one of polymers such as polyethylene and derivatives thereof, hydrocarbons, oils, silicones, siloxanes, organosilicones, quaternary ammonium compounds, ester-functional quaternary ammonium compounds, fluorocarbons, substituted C₁₀-C₂₂ alkanes, substituted C₁₀-C₂₂ alkenes, in certain embodiments, the substituted C₁₀-C₂₂ alkenes may be derivatives of fatty alcohols, polyols, derivatives of polyols such as esters and ethers, sugar derivatives such as ethers and esters or mixtures thereof.

In one embodiment, the surface treating composition can comprise a microemulsion and/or a macroemulsion of a surface treating agent in water. In such an example, the concentration of the surface treating agent within the surface treating composition may be from about 3% to about 60% and/or from about 4% to about 50% and/or from about 5% to about 40%. Nonlimiting examples of such microemulsions are commercially available from Wacker Chemie AG München, Germany (MR1003, MR103, MR102). A nonlimiting example of such a macroemulsion is commercially available from General Electric Silicones, Wilton, Conn. (CM849).

Emollient

In some embodiments of the substrates, emollients may (1) improve the glide of the substrate on the skin, by enhancing the lubrication and thus decreasing the abrasion of the skin, (2) hydrate the residues (for example, fecal residues or dried urine residues), thus enhancing their removal from the skin, (3) hydrate the skin, thus reducing its dryness and irritation while improving its flexibility under the wiping movement, and (4) protect the skin from later irritation (for example, caused by the friction of underwear) as the emollient is deposited onto the skin and remains at its surface as a thin protective layer.

In one embodiment, emollients may be silicone based. Silicone-based emollients may be organo-silicone based polymers with repeating siloxane (Si—O) units. Silicone-based emollients of substrate embodiments may be hydrophobic and may exist in a wide range of possible molecular weights. They may include linear, cyclic and cross-linked varieties. Silicone oils may be chemically inert and may have a high flash point. Due to their low surface tension, silicone oils may be easily spreadable and may have high surface activity. Examples of silicon oil may include: cyclomethicones, dimethicones, phenyl-modified silicones, alkyl-modified silicones, silicones resins and combinations thereof.

Other useful emollients can be unsaturated esters or fatty esters. Examples of unsaturated esters or fatty esters of embodiments include: caprylic capric triglycerides in combination with Bis-PEG/PPG-16/16 PEG/PPG-16/16 dimethicone and C12-C15 alkylbenzoate and combinations thereof.

The amount of emollient that can be included in the lotion composition will depend on a variety of factors, including the particular emollient involved, the lotion-like benefits desired, and the other components in the lotion composition. It has been found that compositions with low or very low emollient content are best suited. The emollient content of the composition is from about 0.001% to less than about 5%, from about 0.001% to less than about 3%, from about 0.001% to less than about 2.5% and from about 0.001% to less than about 1.5% (all % are weight/weight % of the emollient in the composition).

A relatively low surface tension may act more efficiently in the composition. Surface tension lower than about 35 mN/m, or even lower than about 25 mN/m. In certain embodiments, the emollient may have a medium to low polarity. Also, the emollient of an embodiment may have a solubility parameter between about 5 and about 12, or even between about 5 and about 9. The basic reference of the evaluation of surface tension, polarity, viscosity and spreadability of emollient can be found under Dietz, T., Basic properties of cosmetic oils and their relevance to emulsion preparations. SÖFW-Journal, July 1999, pages 1-7.

Emulsifier/Surfactant

The composition may also include an emulsifier such as those forming oil-in-water emulsions. The emulsifier can be a mixture of chemical compounds and include surfactants. The preferred emulsifiers are those acting as well as a surfactant. For the purpose of this document, the terms emulsifiers and surfactants are thereafter used interchangeably. The emulsifier may be a polymeric emulsifier or a non polymeric one.

The emulsifier may be employed in an amount effective to emulsify the emollient and/or any other non-water-soluble oils that may be present in the composition, such as an amount ranging from about 0.5%, 1%, or 4% to about 0.001%, 0.01%, or 0.02% (based on the weight emulsifiers over the weight of the composition). Mixtures of emulsifiers may be used.

Emulsifiers for use in some embodiments may be selected from the group of alkylpolylglucosides, decylpolyglucoside, fatty alcohol or alkoxylated fatty alcohol phosphate esters (e.g., trilaureth-4 phosphate), sodium trideceth-3 carboxylate, or a mixture of caprylic capric triglyceride and Bis-PEG/PPG-16/16 PEG/PPG-16/16 dimethicone, polysorbate 20, and combinations thereof.

Rheology Modifier

Rheology modifiers are compounds that increase the viscosity of the composition at lower temperatures as well as at process temperatures. Each of these materials may also provide “structure” to the compositions to prevent settling out (separation) of insoluble and partially soluble components. Other components or additives of the compositions may affect the temperature viscosity/rheology of the compositions.

In addition to stabilizing the suspension of insoluble and partially soluble components, the rheology modifiers of the invention may also help to stabilize the composition on the substrate and enhance the transfer of lotion to the skin. The wiping movement may increase the shear and pressure therefore decreasing the viscosity of the lotion and enabling a better transfer to the skin as well as a better lubrication effect.

Additionally, the rheology modifier may help to preserve a homogeneous distribution of the composition within a stack of substrates. Any composition that is in fluid form has a tendency to migrate to the lower part of the wipes stack during prolonged storage. This effect creates an upper zone of the stack having less composition than the bottom part.

Preferred rheology modifiers may exhibit low initial viscosity and high yield. Particularly suited are rheology modifiers such as, but not limited to:

Blends of material as are available from Uniqema GmbH&Co. KG, of Emmerich, Germany under the trade name ARLATONE. For instance, ARLATONE V-175 which is a blend of sucrose palmitate, glyceryl stearate, glyceryl stearate citrate, sucrose, mannan, and xanthan gum and Arlatone V-100 which is a blend of steareth-100, steareth-2, glyceryl stearate citrate, sucrose, mannan and xanthan gum.

Blends of materials as are available from Seppic France of Paris, France as SIMULGEL. For example, SIMULGEL NS which comprises a blend of hydroxyethylacrylate/sodium acryloyldimethyl taurate copolymer and squalane and polysorbate 60, sodium acrylate/sodium acryloyldimethyltaurate copolymer and polyisobutene and caprylyl capryl glucoside, acrylate copolymers, such as but not limited to acrylates/acrylamide copolymers, mineral oil, and polysorbate 85.

Acrylate homopolymers, acrylate crosspolymers, such as but not limited to, Acrylate/C10-30 Alkyl Acrylate crosspolymers, carbomers, such as but not limited to acrylic acid cross linked with one or more allyl ether, such as but not limited to allyl ethers of pentaerythritol, allyl ethers of sucrose, allyl ethers of propylene, and combinations thereof as are available are available as the Carbopol® 900 series from Noveon, Inc. of Cleveland, Ohio (e.g., Carbopol® 954).

Naturally occurring polymers such as xanthan gum, galactoarabinan and other polysaccharides.

Combinations of the above rheology modifiers.

Examples, of commercially available rheology modifiers include but are not limited to, Ultrez-10, a carbomer, and Pemulen TR-2, acrylate crosspolymers, both of which are available from Noveon, Cleveland Ohio, and Keltrol, a xanthan gum, available from CP Kelco San Diego Calif.

Rheology modifiers imparting a low viscosity may be used. Low viscosity is understood to mean viscosity of less than about 10,000 cps at about 25 degrees Celsius of a 1% aqueous solution. The viscosity may be less than about 5,000 cps under the same conditions. Further, the viscosity may be less than about 2000 cps or even less than about 1,000 cps. Other characteristics of emulsifiers may include high polarity and a non-ionic nature.

Rheology modifiers, when present may be used at a weight/weight % (w/w) from about 0.01%, 0.015%, or 0.02% to about 1%, 2%, or 3%.

Preservative

The need to control microbiological growth in personal care products is known to be particularly acute in water based products such as oil-in-water emulsions and in pre-impregnated substrates such as baby wipes. The composition may comprise a preservative or a combination of preservatives acting together as a preservative system. Preservatives and preservative systems are used interchangeably in the present document to indicate one unique or a combination of preservative compounds. A preservative is understood to be a chemical or natural compound or a combination of compounds reducing the growth of microorganisms, thus enabling a longer shelf life for the pack of wipes (opened or not opened) as well as creating an environment with reduced growth of microorganisms when transferred to the skin during the wiping process.

Preservatives of certain embodiments can be defined by 2 key characteristics: (i) activity against a large spectrum of microorganisms, that may include bacteria and/or molds and/or yeast, or all three categories of microorganisms together and (2) killing efficacy and/or the efficacy to reduce the growth rate at a concentration as low as possible.

The spectrum of activity of the preservative of embodiments may include bacteria, molds and yeast. Ideally, each of such microorganisms are killed by the preservative. Another mode of action to be contemplated is the reduction of the growth rate of the microorganisms without active killing. Both actions however result in a drastic reduction of the population of microorganisms.

Suitable materials include, but are not limited to a methylol compound, or its equivalent, an iodopropynyl compound and mixtures thereof. Methylol compounds release a low level of formaldehyde when in water solution that has effective preservative activity. Exemplary methylol compounds include but are not limited to: diazolidinyl urea (GERMALL® II as is available from International Specialty Products of Wayne, N.J.) N-[1,3-bis(hydroxy-methyl)-2,5-dioxo-4-imidazolidinyl]-N,N′-bis(hydroxymethyl)urea, imidurea (GERMALL® 115 as is available from International Specialty Products of Wayne, N.J.), 1,1-methylene bis[3-[3-(hydroxymethyl)-2,5-dioxo-4-imidazolidinyl]urea]; 1,3-dimethylol-5,5-dimethyl hydantoin (DMDMH), sodium hydroxymethyl glycinate (SUTTOCIDE® A as is available from International Specialty Products of Wayne, N.J.), and glycine anhydride dimethylol (GADM). Methylol compounds can be effectively used at concentrations (100% active basis) between about 0.025% and about 0.50%. A preferred concentration (100% basis) is about 0.075%. The iodopropynyl compound provides antifungal activity. An exemplary material is iodopropynyl butyl carbamate as is available from Clariant UK, Ltd. of Leeds, The United Kingdom as NIPACIDE IPBC. A particularly preferred material is 3-iodo-2-propynylbutylcarbamate. Iodopropynyl compounds can be used effectively at a concentration between about 0% and about 0.05%. A preferred concentration is about 0.009%. A particularly preferred preservative system of this type comprise a blend of a methylol compound at a concentration of about 0.075% and a iodopropynyl compound at a concentration of about 0.009%.

In another embodiment, the preservative system may comprise simple aromatic alcohols (e.g., benzyl alcohol). Materials of this type have effective anti bacterial activity. Benzyl alcohol is available from Symrise, Inc. of Teterboro, N.J.

In another embodiment, the preservative may be a paraben antimicrobial selected from the group consisting of methylparaben, ethylparaben, propylparaben, butylparaben, isobutylparaben or combinations thereof.

In another embodiment, the preservative may be a low-pH acid and/or buffer-system to maintain a pH less than about 4.5.

Chelators (e.g., ethylenediamine tetraacetic acid and its salts) may also be used in preservative systems as a potentiator for other preservative ingredients.

The preservative composition can also provide a broad anti-microbial effect without the use of formaldehyde donor derived products.

Optional Components of the Composition

The composition may optionally include adjunct ingredients. Possible adjunct ingredients may be selected from a wide range of additional ingredients such as, but not limited to soothing agents, perfumes and fragrances, texturizers, colorants, and medically active ingredients, in particular healing actives and skin protectants.

Soothing agents are compounds having the ability to reduce the irritation or stinging/burning/itching effect of some chemicals. Soothing agents can be of a variety of chemical classes. Soothing agents can have a variety of modes of action to neutralize the effects of the skin irritants, especially for paraben based preservative systems. For example antioxidants can be soothing agents for oxidants. Buffers can be soothing agents neutralizing the stinging effect on skin of acids or bases. It is to be noted that emollients can also be soothing agents. Soothing agents that act against the stinging/irritation effect of some preservatives are preferred. Those soothing agents can be emollients or surfactants helping, for example, the solubilization or the micellization of the preservatives.

Optional soothing agents may be (a) ethoxylated surface active compounds, those having an ethoxylation number below about 60, (b) polymers, polyvinylpyrrolidone (PVP) and/or N-vinylcaprolactam homopolymer (PVC), and (c) phospholipids, phospholipids complexed with other functional ingredients as e.g., fatty acids, organosilicones.

The soothing agents may be selected from the group comprising PEG-40 hydrogenated castor oil, sorbitan isostearate, isoceteth-20, sorbeth-30, sorbitan monooleate, coceth-7, PPG-1-PEG-9 lauryl glycol ether, PEG-45 palm kernel glycerides, PEG-20 almond glycerides, PEG-7 hydrogenated castor oil, PEG-50 hydrogenated castor oil, PEG-30 castor oil, PEG-24 hydrogenated lanolin, PEG-20 hydrogenated lanolin, PEG-6 caprylicicapric glycerides, PPG-1 PEG-9 lauryl glycol ether, lauryl glucoside polyglyceryl-2 dipolyhydroxystearate, sodium glutamate, polyvinylpyrrolidone, N-vinylcaprolactam homopolymer, sodium coco PG-dimonium chloride phosphate, linoleamidopropyl PG-dimonium chloride phosphate, dodium borageamidopropyl PG-dimonium chloride phosphate, N-linoleamidopropyl PG-dimonium chloride phosphate dimethicone, cocamidopropyl PG-dimonium chloride phosphate, stearamidopropyl PG-dimonium chloride phosphate and stearamidopropyl PG-dimonium chloride phosphate (and) cetyl alcohol, and combinations thereof. A particularly preferred soothing agent is PEG-40 hydrogenated castor oil as is available from BASF of Ludwigshafen, Germany as Cremophor CO 40.

Representative examples of lotion composition useful in embodiments are given as Examples A-D below.

### Example A

| Component | Amount (% by weight) |
|---|---|
| (1) Disodium EDTA | 0.10 |
| (2) Arlatone-V 175 ™* | 0.80 |
| (3) Decylglycoside | 0.05 |
| (4) Cyclopentasiloxane Dimethiconol | 0.45 |
| (5) 1,2-Propyleneglycol | 1.50 |
| (6) Phenoxyethanol | 0.80 |
| (7) Methylparaben | 0.15 |
| (8) Propylparaben | 0.05 |
| (9) Ethylparaben | 0.05 |
| (10) PEG-40 Hydrogenated Castor Oil | 0.80 |
| (11) Perfume | 0.05 |
| (12) Purified water | Balance |
| Total | 100 |

*Arlatone-V 175 ™ comprises sucrose palmitate, glyceryl stearate, glyceryl stearate citrate, sucrose, mannan, xanthan gum and is commercialized by Uniqema GmbH&Co. KG 46429 Emmerich, Germany, www.uniqema.com.

### Example B

| | Amount |
|---|---|
| Component | (% by weight) |
| (1) Disodium EDTA | 0.10 |
| (2) Arlatone-V 175 ™* | 0.80 |
| (3) Abil Care 85 ™** | 0.45 |
| (4) Decylglycoside | 0.05 |
| (5) 1,2-Propyleneglycol | 1.50 |
| (6) Sodium benzoate | 0.20 |
| (7) Methylparaben | 0.15 |
| (8) Propyiparaben | 0.05 |
| (9) Ethylparaben | 0.05 |
| (10) PEG-40 Hydrogenated Castor Oil | 0.80 |
| (11) Perfume | 0.05 |
| (12) Purified water | Balance |
| Total | 100.00 |

*Arlatone-V 175 ™ comprises sucrose palmitate, glyceryl stearate, glyceryl stearate citrate, sucrose, mannan, xanthan gum and is commercialized by Uniqema GmbH&Co. KG, 46429 Emmerich, Germany, www.uniqema.com.
**Abil Care 85 ™ comprises Bis-PEG/PPG-16/16 PEG/PPG Dimethicone Caprylic Capric triglyceride and is commercialized by Goldschmidt/Degussa, Goldschmidt AG, 45127 Essen, Germany www.goldschmidt.com.

### Example C

| Component | Amount (% by weight) |
|---|---|
| (1) Disodium EDTA | 0.10 |
| (2) Xanthan Gum | 0.18 |
| (3) Abil Care 85 ™** | 0.10 |
| (4) 1,2-Propyleneglycol | 1.50 |
| (5) Phenoxyethanol | 0.60 |
| (6) Methylparaben | 0.15 |
| (7) Propylparaben | 0.05 |
| (8) Ethylparaben | 0.05 |
| (9) Trilaureth-4 Phosphate | 0.40 |
| (10) PEG-40 Hydrogenated Castor Oil | 0.40 |
| (11) Perfume | 0.07 |
| (12) Purified water | Balance |
| Total | 100.00 |

**Abil Care 85 ™ comprises Bis-PEG/PPG-16/16 PEG/PPG Dimethicone Caprylic Capric triglyceride and is commercialized by Goldschmidt/Degussa, Goldschmidt AG, 45127 Essen, Germany www.goldschmidt.com.

### Example D

| Component | Amount (% by weight) |
|---|---|
| (1) Disodium EDTA | 0.10 |
| (2) Xanthan Gum | 0.18 |
| (3) Abil Care 85 ™** | 0.10 |
| (4) Sodium Benzoate | 0.12 |
| (5) Citric Acid | 0.53 |
| (6) Sodium Citrate | 0.39 |
| (7) Benzyl Alcohol | 0.30 |
| (8) Euxyl PE9010*** | 0.30 |
| (10) PEG-40 Hydrogenated Castor Oil | 0.44 |
| (11) Perfume | 0.07 |
| (12) Purified water | Balance |
| Total | 100 |

**Abil Care 85 ™ comprises Bis-PEG/PPG-16/16 PEG/PPG Dimethicone Caprylic Capric triglyceride and is commercialized by Goldschmidt/Degussa, Goldschmidt AG, 45127 Essen, Germany www.goldschmidt.com.
***Euxyl PE9010 tm comprises a mixture of phenoxyethanol and ethylhexylglycerin and is commercialized by Schulke & Mayr GmbH, Germany.

Method of Making Molded Fibrous Structure

Generally, the process for making a fibrous structure may be described in terms of initially forming a fibrous web having a plurality of synthetic fibers, a plurality of natural fibers, or a combination thereof. Layered deposition of the fibers, synthetic and natural, are also contemplated. In an embodiment, the fibrous web can be formed in any fashion and may be any nonwoven web suitable for use in a hydromolding process. The fibrous web may consist of any web, mat, or batt of loose fibers disposed in any relationship with one another in any degree of alignment, such as might be produced by carding, air-laying, spunmelting (including meltblowing and spunlaying), coforming and the like.

In an embodiment, a fibrous web may be produced by conducting the carding, spunmelting, spunlaying, meltblowing, coforming, or air-laying or other bonding processes concurrently with the fibers contacting a forming member. In addition, the process may involve subjecting the fibrous web to a hydroentanglement process while the fibrous web is in contact with the forming member. The hydroentanglement process (also known as spunlacing or spunbonding) is a known process of producing nonwoven webs, and involves laying down a matrix of fibers, for example as a carded web or an air-laid web, and entangling the fibers to form a coherent web. Entangling is typically accomplished by impinging the matrix of fibers with high pressure liquid (typically water) from one or more suitably-placed water jets. The pressure of the liquid jets, as well as the orifice size and the energy imparted to the fibrous structure by the water jets, may be the same as those of a conventional hydroentangling process. Typical entanglement energy is about 0.1 kwh/kg. Optionally, other fluids can be used as the impinging medium, such as compressed air. The fibers of the web are entangled, but not physically bonded one to another. The fibers of a hydroentangled web, therefore, have more freedom of movement than fibers of webs formed by thermal or chemical bonding. Particularly when lubricated by wetting, as in a pre-moistened wet wipe, such spunlaced webs provide webs having very low bending torques and low moduli, thereby providing softness and suppleness.

Additional information on hydroentanglement can be found in U.S. Pat. Nos. 3,485,706 issued on Dec. 23, 1969, to Evans; 3,800,364 issued on Apr. 2, 1974, to Kalwaites; 3,917,785 issued on Nov. 4, 1975, to Kalwaites; 4,379,799 issued on Apr. 12, 1983, to Holmes; 4,665,597 issued on May 19, 1987, to Suzuki; 4,718,152 issued on Jan. 12, 1988, to Suzuki; 4,868,958 issued on Sep. 26, 1989, to Suzuki; 5,115,544 issued on May 26, 1992, to Widen; and 6,361,784 issued on Mar. 26, 2002, to Brennan. After the fibrous web has been formed, it can be subjected to additional process steps, such as, for example, hydromolding (also known as molding, hydro-embossing, hydraulic needle-punching, etc.). The resulting molded fibrous structure may be processed in any method to covert the molded fibrous structure to a substrate suitable for use as a wipe. This may include, but is not limited to, slitting, cutting, perforating, folding, stacking, interleaving, lotioning and combinations thereof.

Hydromolding, as may be applied to substrates useful as wipes, which may include a number of decorative patterns. Such patterns may include regular arrays of small geometric shapes (such as, for example, circles, squares, rectangles, ovals, triangles, octagons, tear drops, droplets, etc.) regular repeating patterns of lines, and curves, images of animals, etc.

Other beneficial physical characteristics may be imparted to the fibrous web by hydromolding. Specifically, hydromolding a fibrous web may have an effect on the interfacial pore size distribution occurring between adjacent wipes in a stack of wet wipes, and thereby may have an effect on the dispensing forces for individual wipes when dispensed from a package.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “90 degrees” is intended to mean “about 90 degrees”.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A fibrous web structure comprising:
a first broad outer macroscopic surface; and
a second broad outer macroscopic surface opposite the first broad outer macroscopic surface thereby defining an absorbent fibrous region between the first and second broad outer macroscopic surfaces, the fibrous web structure extending in a longitudinal direction, the absorbent fibrous region having a thickness extending in a transverse direction that is perpendicular to the longitudinal direction;
a formed fibrous feature defining a cavity in the first broad outer macroscopic surface, the formed fibrous feature having a wave portion formed of the fibrous region extending into a mouth of the cavity and a pocket defined by the cavity extending beneath the wave portion such that the wave portion overhangs the pocket, the formed fibrous feature comprising a leading wall and a trailing wall, wherein the leading wall faces the wave portion and the trailing wall forms the wave portion.

2. The fibrous web structure of claim 1, wherein the cavity has a first width measured in the longitudinal direction at the mouth of the cavity and a second width measured in the longitudinal direction below the mouth of the cavity, the second width being greater than the first width.

3. The fibrous web structure of claim 1, wherein the formed fibrous feature extends in a lateral direction continuously between opposite sides of the first broad outer macroscopic surface.

4. The fibrous web structure of claim 1 comprising a plurality of formed fibrous features, each formed fibrous feature defining a cavity in the first broad outer macroscopic surface.

5. The fibrous web structure of claim 1 further comprising a lotion.

6. A fibrous web structure comprising:
a first broad outer macroscopic surface; and
a second broad outer macroscopic surface opposite the first broad outer macroscopic surface thereby defining an absorbent fibrous region between the first and second broad outer macroscopic surfaces, the fibrous web structure extending in a longitudinal direction, the absorbent fibrous region having a thickness extending in a transverse direction that is perpendicular to the longitudinal direction;
a formed fibrous feature preform defining a cavity preform in the first broad outer macroscopic surface, the formed fibrous feature preform comprising a fibrous projection preform that includes a leading wall, a trailing wall and a longitudinal wall, wherein the longitudinal wall lies at an angle that is oblique to the first broad outer macroscopic surface.

7. The fibrous web structure of claim 6, wherein the leading wall and the trailing wall have different heights measured in the transverse direction.

8. The fibrous web structure of claim 6, wherein a ratio of maximum height of the cavity preform to width at the mouth of the cavity preform is at least about 1.

9. A method of forming a fibrous web structure including a first broad outer macroscopic surface and a second broad outer macroscopic surface opposite the first broad outer macroscopic surface thereby defining an absorbent fibrous region between the first and second broad outer macroscopic surfaces, the fibrous web structure extending in a longitudinal direction, the method comprising:
forming a formed fibrous feature preform defining a cavity preform in the first broad outer macroscopic; and
forming a final formed fibrous feature defining a final cavity in the first broad outer macroscopic surface, the final formed fibrous feature having a wave portion formed of the fibrous region extending in the longitudinal direction into the final cavity and a pocket defined by the final cavity extending in the longitudinal direction beneath the wave portion such that the wave portion overhangs the pocket.

10. The method of claim 9 comprising compressing the formed fibrous feature preform thereby buckling the formed fibrous feature preform and forming the wave portion formed of the absorbent fibrous region.
